(19) United States
(12) Patent Application Publication
(10) Pub. No.: US 2025/0084462 A1
(43) Pub. Date: Mar. 13, 2025

(54) SYSTEMS AND METHODS FOR DETECTION OF ELECTROCHEMICALLY LABELED OLIGONUCLEOTIDES

(71) Applicant: SEEK LABS, INC., Salt Lake City, UT (US)

(72) Inventors: Deric Holden, Salt Lake City, UT (US); Elijah Allen, Salt Lake City, UT (US); Jared Bauer, Salt Lake City, UT (US)

(21) Appl. No.: 18/466,390

(22) Filed: Sep. 13, 2023

(51) Int. Cl. C12Q 1/6825

(52) U.S. Cl. CPC C12Q 1/6825

(57) ABSTRACT

Methods, systems, and compositions for probeless detection of oligonucleotides featuring sensors comprising electrochemically tagged DNA or RNA oligonucleotide primers adhered to any appropriate electrode surface, such as but not limited to carbon screen printed electrodes. A potentiostat can be used to produce a signal, the strength of which may depend on the length of the tagged oligonucleotide primers and concentration thereof.

### FIELD OF THE INVENTION

The present invention features methods, systems, and compositions for probeless detection of electrochemically labeled oligonucleotides.

### BACKGROUND OF THE INVENTION

Electrochemical sensors, or genosensors, can be used to detect target oligonucleotides (e.g., DNA, RNA, single-stranded oligonucleotides, double-stranded DNA, etc.). Current electrochemical sensors rely on bonding probes, e.g., oligonucleotide probes complementary to the target oligonucleotides, to a gold surface. Manufacturing of these types of sensors can be difficult and time consuming to produce and is difficult to scale. They also present challenges related to detecting non-specific oligonucleotides.

### BRIEF SUMMARY OF THE INVENTION

The present invention provides methods, systems, and compositions for preparing oligonucleotides for detection, or for detecting oligonucleotides. The methods, systems, and compositions can be used for a variety of applications, such as but not limited to determining if a particular sample contains amplified oligonucleotides after amplification. For example, the methods, systems, and compositions can be used to compare the amount of labeled oligonucleotides in a sample prior to amplification to the amount of labeled oligonucleotides in a sample after amplification to determine if amplification occurred. The methods and systems can also be used for other comparative purposes, for example, for comparing an amount of labeled DNA in a first sample (or a first portion of a sample) with an amount of labeled DNA in a second sample (or a second portion of a sample). Embodiments of the present invention can be freely combined with each other if they are not mutually exclusive.

Without wishing to limit the present invention to any theory or mechanism, it is believed that the methods, systems, and compositions of the present invention are advantageous because they feature probeless detection, e.g., the methods, systems, and compositions do not require the covalent bonding of probes to a surface. As a non-limiting example, the present invention features probeless detection that utilizes electrochemically labeled DNA or RNA oligonucleotides, e.g., primers, adhered (e.g., non-covalently) to any appropriate electrode surface, such as but not limited to carbon SPE. These sensors (e.g., electrochemically labeled DNA, e.g., ssDNA, or RNA adhered to the electrode surface) may be created by depositing a volume (e.g., 15-100 ul as an example, although the volume may depend on the size of the surface and the concentration of oligonucleotides) of tagged oligonucleotide onto the surface and allowing it to adhere for a period of time (e.g., 30 seconds to 30 minutes, which may depend on various factors such as the needs of the test). Square wave voltammetry may be run with the labeled oligonucleotide solution still on the surface of the electrode. The tagged (electrochemically labeled) oligonucleotides result in a signal. The signal may change strength depending on the length of the labeled oligonucleotide, abundance of the labeled oligonucleotide, etc.

As used herein, the term “adhere” is a general term that refers to the relationship between the oligonucleotides and the surface, wherein a non-covalent, attractive force exists between the oligonucleotides and the surface. In some embodiments, the force that attracts the oligonucleotides to the surface is Van der Waals force. However, the present invention may not be limited to Van der Waals force as being the only mechanism that creates the attraction between the oligonucleotides and the surface. The attractive force between the oligonucleotides and the surface is not a covalent linkage.

This system relies on amplification techniques to create a detectable sample. A solution filled with short DNA primers can be used to establish a strong baseline peak. The primers would then be amplified, reducing the concentration of the short primers and heavily increasing the concentration of longer DNA amplicons. The amplified solution can then be used on a sensor, and a decrease in the signal indicates that the primer concentration has been decreased and amplification has occurred.

For example, the present invention features a system comprising a surface and a plurality of oligonucleotides adhered to the surface, wherein at least a portion of the oligonucleotides are labeled with an electrochemical label. The system may be used for applications such as oligonucleotide detection, e.g., for detecting amplification of a target oligonucleotide. In some embodiments, the oligonucleotides comprise DNA or RNA. In some embodiments, the oligonucleotides are single-stranded. In some embodiments, the oligonucleotides are double-stranded.

In some embodiments, the surface comprises a carbon screen printed electrode, platinum, a glassy carbon electrode, a glass strip, a gold electrode, a carbon screen printed electrode modified with carbon nanotubes, a carbon screen printed electrode modified with a metal nanoparticle, platinum modified with carbon nanotubes, platinum modified with a metal nanoparticle, a glassy carbon electrode modified with carbon nanotubes, a glassy carbon electrode modified with a metal nanoparticle, a glass strip modified with carbon nanotubes, a glass strip modified with a metal nanoparticle, a gold electrode modified with carbon nanotubes, or a gold electrode modified with a metal nanoparticle. In some embodiments, the metal nanoparticle comprises gold, silver, iron, cobalt, platinum, carbon, or copper.

In some embodiments, the electrochemical label is selected from methylene blue, methylene violet, ruthenium hexamine, ferrocene, anthraquinone, an anthraquinone derivative, a metal nanoparticle (e.g., gold, silver, iron, cobalt, platinum, carbon, or copper, other appropriate metal, a combination thereof), or a combination thereof.

In some embodiments, the surface is free of a single-stranded probe composition comprising single-stranded oligonucleotides complementary to the oligonucleotides labeled with the electrochemical label.

In some embodiments, the system further comprises a potentiostat for detecting oligonucleotides labeled with the electrochemical label adhered to the surface.

As is described herein, the present invention also includes a system comprising a carbon screen printed electrode (SPE) surface and a plurality of single-stranded DNA oligonucleotides adhered, e.g., via Van der Waals forces, to the carbon SPE surface, wherein at least a portion of the single-stranded DNA oligonucleotides are labeled with an electrochemical label. In some embodiments, the electrochemical label is selected from methylene blue, methylene violet, ruthenium hexamine, ferrocene, anthraquinone, an anthraquinone derivative, a metal nanoparticle (e.g., gold, silver, iron, cobalt, platinum, carbon, copper, another appropriate metal, or a combination thereof), or a combination thereof. The system may be used for applications such as oligonucleotide detection, e.g., for detecting amplification of a target oligonucleotide. In some embodiments, the oligonucleotides comprise DNA, e.g., ssDNA or RNA. In some embodiments, the oligonucleotides are single-stranded. In some embodiments, the oligonucleotides are double-stranded.

In some embodiments, the surface is free of a single-stranded probe composition comprising single-stranded oligonucleotides complementary to the oligonucleotides labeled with the electrochemical label. In some embodiments, the system further comprises a potentiostat for detecting single-stranded DNA oligonucleotides labeled with the electrochemical label adhered to the surface.

The present invention also features a kit comprising a surface for adhering a plurality of oligonucleotides and an electrochemical label for labeling the plurality of oligonucleotides. In some embodiments, the surface is selected from: a carbon screen printed electrode, platinum, a glassy carbon electrode, a glass strip, a gold electrode, a carbon screen printed electrode modified with carbon nanotubes, a carbon screen printed electrode modified with a metal nanoparticle, platinum modified with carbon nanotubes, platinum modified with a metal nanoparticle, a glassy carbon electrode modified with carbon nanotubes, a glassy carbon electrode modified with a metal nanoparticle, a glass strip modified with carbon nanotubes, a glass strip modified with a metal nanoparticle, a gold electrode modified with carbon nanotubes, or a gold electrode modified with a metal nanoparticle. In some embodiments, the metal nanoparticle comprises gold, silver, iron, cobalt, platinum, carbon, or copper.

In some embodiments, the electrochemical label is selected from methylene blue, methylene violet, ruthenium hexamine, ferrocene, anthraquinone, an anthraquinone derivative, a metal nanoparticle (e.g., gold, silver, iron, cobalt, platinum, carbon, or copper, other appropriate metal, a combination thereof), or a combination thereof.

The present invention also features a method of preparing a surface for oligonucleotide detection. In some embodiments, the method comprises incubating a surface with a plurality of oligonucleotides for a period of time, wherein at least a portion of the oligonucleotides are labeled with an electrochemical label. In some embodiments, the surface is incubated with a solution of the plurality of oligonucleotides (a portion thereof are electrochemically labeled) at a ratio of at least 20 μL of solution per 10 mm² of the surface. In some embodiments, the surface is incubated with a solution of the plurality of oligonucleotides at a ratio of at least 10 μL of solution per 10 mm² of the surface. In some embodiments, the plurality of oligonucleotides is at a concentration of at least 10 nM. In some embodiments, the plurality of oligonucleotides is at a concentration of at least 40 nM. In some embodiments, the plurality of oligonucleotides is at a concentration of at least 100 nM. In some embodiments, the period of time is at least 30 seconds. In some embodiments, the period of time is from 30-60 seconds. In some embodiments, the period of time is from 1 to 10 minutes. In some embodiments, the period of time is from 10 to 30 minutes. In some embodiments, the period of time is from 30 to 60 minutes. In some embodiments, the period of time is from 60 to 120 minutes.

In some embodiments, the surface is selected from: a carbon screen printed electrode, platinum, a glassy carbon electrode, a glass strip, a gold electrode, a carbon screen printed electrode modified with carbon nanotubes, a carbon screen printed electrode modified with a metal nanoparticle, platinum modified with carbon nanotubes, platinum modified with a metal nanoparticle, a glassy carbon electrode modified with carbon nanotubes, a glassy carbon electrode modified with a metal nanoparticle, a glass strip modified with carbon nanotubes, a glass strip modified with a metal nanoparticle, a gold electrode modified with carbon nanotubes, or a gold electrode modified with a metal nanoparticle. In some embodiments, the metal nanoparticle comprises gold, silver, iron, cobalt, platinum, carbon, or copper.

In some embodiments, the electrochemical label is selected from methylene blue, methylene violet, ruthenium hexamine, ferrocene, anthraquinone, an anthraquinone derivative, a metal nanoparticle (e.g., gold, silver, iron, cobalt, platinum, carbon, or copper, other appropriate metal, a combination thereof), or a combination thereof.

The present invention also features a method of detecting an oligonucleotide. In some embodiments, the method comprises incubating a surface with a volume of a solution comprising a plurality of oligonucleotides for a period of time, wherein at least a portion of the oligonucleotides are labeled with an electrochemical label, and detecting an amount of electrochemically labeled oligonucleotides adhered to the surface using a potentiostatic system which detects the electrochemical labels of the oligonucleotides. In some embodiments, the potentiostatic system is a square wave voltammetry system.

In some embodiments, the surface is selected from: a carbon screen printed electrode, platinum, a glassy carbon electrode, a glass strip, a gold electrode, a carbon screen printed electrode modified with carbon nanotubes, a carbon screen printed electrode modified with a metal nanoparticle, platinum modified with carbon nanotubes, platinum modified with a metal nanoparticle, a glassy carbon electrode modified with carbon nanotubes, a glassy carbon electrode modified with a metal nanoparticle, a glass strip modified with carbon nanotubes, a glass strip modified with a metal nanoparticle, a gold electrode modified with carbon nanotubes, or a gold electrode modified with a metal nanoparticle. In some embodiments, the metal nanoparticle comprises gold, silver, iron, cobalt, platinum, carbon, or copper.

In some embodiments, the electrochemical label is selected from methylene blue, methylene violet, ruthenium hexamine, ferrocene, anthraquinone, an anthraquinone derivative, a metal nanoparticle (e.g., gold, silver, iron, cobalt, platinum, carbon, or copper, other appropriate metal, a combination thereof), or a combination thereof.

The present invention also features a method of detecting amplification of a target oligonucleotide. In some embodiments, the method comprises incubating a first surface with a volume of a first solution comprising a plurality of single-stranded, electrochemically labeled primers specific for the target oligonucleotide for a period of time and detecting an amount of single-stranded, electrochemically labeled primers in the first solution adhered to the first surface using a potentiostatic system, which detects the electrochemical labels of the single-stranded, electrochemically labeled primers. In some embodiments, the method further comprises incubating a second surface (the second surface having the same composition as the first surface) with a volume of a second solution for a period of time, the second solution comprising one or both of: (i) a plurality of single-stranded, electrochemically labeled primers specific for the target oligonucleotide, and/or (ii) target oligonucleotide amplicons derived from the plurality of single-stranded, electrochemically labeled primers specific for the target oligonucleotide; and detecting an amount of single-stranded, electrochemically labeled primers in the second solution adhered to the second surface using a potentiostatic system, which detects the electrochemical labels of the single-stranded, electrochemically labeled primers. The method may further comprise comparing the amount of single-stranded, electrochemically labeled primers in the first solution adhered to the first surface with the amount of single-stranded, electrochemically labeled primers in the second solution adhered to the second surface, wherein if the amount of single-stranded, electrochemically labeled primers adhered to the second surface is less than the amount of single-stranded, electrochemically labeled primers adhered to the first surface, then the second solution comprises amplified target oligonucleotides.

In some embodiments, the potentiostatic system is a square wave voltammetry system. In some embodiments, the potentiostatic system is a cyclic voltammetry system, a chronoamperometry system, or an electrochemical impedance spectroscopy system.

In some embodiments, the single-stranded primers are from 10-50 nucleotides in length. In some embodiments, the amplicons are from 80 to 120 nucleotides in length.

In some embodiments, the first surface is selected from: a carbon screen printed electrode, platinum, a glassy carbon electrode, a glass strip, a gold electrode, a carbon screen printed electrode modified with carbon nanotubes, a carbon screen printed electrode modified with a metal nanoparticle, platinum modified with carbon nanotubes, platinum modified with a metal nanoparticle, a glassy carbon electrode modified with carbon nanotubes, a glassy carbon electrode modified with a metal nanoparticle, a glass strip modified with carbon nanotubes, a glass strip modified with a metal nanoparticle, a gold electrode modified with carbon nanotubes, or a gold electrode modified with a metal nanoparticle. In some embodiments, the metal nanoparticle comprises gold, silver, iron, cobalt, platinum, carbon, or copper.

In some embodiments, the electrochemical label is selected from methylene blue, methylene violet, ruthenium hexamine, ferrocene, anthraquinone, an anthraquinone derivative, a metal nanoparticle (e.g., gold, silver, iron, cobalt, platinum, carbon, or copper, other appropriate metal, a combination thereof), or a combination thereof.

The present invention also features a method of preparing a surface for oligonucleotide detection. In some embodiments, the method comprises incubating a surface with a plurality of oligonucleotides for a period of time, wherein at least a portion of the oligonucleotides are labeled with an electrochemical label. In some embodiments, at least 10% of the oligonucleotides in the plurality of oligonucleotides adhere to the surface within the period of time. In some embodiments, at least 20% of the oligonucleotides in the plurality of oligonucleotides adhere to the surface within the period of time. In some embodiments, at least 25% of the oligonucleotides in the plurality of oligonucleotides adhere to the surface within the period of time. In some embodiments, at least 30% of the oligonucleotides in the plurality of oligonucleotides adhere to the surface within the period of time. In some embodiments, at least 40% of the oligonucleotides in the plurality of oligonucleotides adhere to the surface within the period of time. In some embodiments, at least 50% of the oligonucleotides in the plurality of oligonucleotides adhere to the surface within the period of time.

The present invention also features a system comprising: a surface selected from: a carbon screen printed electrode, platinum, a glassy carbon electrode, a glass strip, a gold electrode, a carbon screen printed electrode modified with carbon nanotubes, a carbon screen printed electrode modified with a metal nanoparticle, platinum modified with carbon nanotubes, platinum modified with a metal nanoparticle, a glassy carbon electrode modified with carbon nanotubes, a glassy carbon electrode modified with a metal nanoparticle, a glass strip modified with carbon nanotubes, a glass strip modified with a metal nanoparticle, a gold electrode modified with carbon nanotubes, or a gold electrode modified with a metal nanoparticle; and a plurality of single-stranded DNA oligonucleotides adhered to the carbon SPE surface via Van der Waals forces, wherein at least a portion of the single-stranded DNA oligonucleotides are labeled with an electrochemical label.

In some embodiments, the single-stranded DNA oligonucleotides are single-stranded primers or are single-stranded DNA amplicons comprising single-stranded primers. In some embodiments, the single-stranded primers are from 10-50 nucleotides in length. In some embodiments, the amplicons are from 80 to 120 nucleotides in length. In some embodiments, the metal nanoparticle comprises gold, silver, iron, cobalt, platinum, carbon, or copper. In some embodiments, the electrochemical label is selected from methylene blue, methylene violet, ruthenium hexamine, ferrocene, anthraquinone, an anthraquinone derivative, a metal nanoparticle, or a combination thereof.

The present invention also features a system comprising a carbon screen printed electrode and a plurality of single-stranded DNA oligonucleotides adhered to the carbon SPE surface via Van der Waals forces, wherein at least a portion of the single-stranded DNA oligonucleotides are labeled with an electrochemical label. In some embodiments, the single-stranded DNA oligonucleotides are single-stranded primers, or are single-stranded DNA amplicons comprising single-stranded primers. In some embodiments, the single-stranded primers are from 10-50 nucleotides in length. In some embodiments, the amplicons are from 80 to 120 nucleotides in length. In some embodiments, the electrochemical label is selected from methylene blue, methylene violet, ruthenium hexamine, ferrocene, anthraquinone, an anthraquinone derivative, a metal nanoparticle, or a combination thereof.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows a schematic diagram of how the methods and systems described herein function. For example, short oligonucleotides labeled with an electrochemical label, e.g., methylene blue, produce a strong signal. During the amplification process, the short oligonucleotides are converted to longer amplicons. These longer amplicons interact differently with the substrate, yielding a lower signal, which is interpreted as positive detection and verification of DNA amplification. In some embodiments, a control is performed by placing non-tagged targets and/or genomic DNA onto the sensor, which yields no signal.

FIG. 2 shows the performance of different materials that may be used as a surface for the systems and devices described herein. Testing was done on material such as gold electrodes, glassy carbon electrodes, metrohm carbon screen printed electrodes (CSPE), and glass test strips. As shown in FIG. 2, some of the surfaces showed detection for deposition times as short as 30 seconds, which is a significant improvement over the hour deposition time required for both probe and target deposition on genosensors.

FIG. 3 shows a non-limiting example of the set up for the systems and devices described herein.

### DETAILED DESCRIPTION OF THE INVENTION

For purposes of summarizing the disclosure, certain aspects, advantages, and novel features of the disclosure are described herein. It is to be understood that not necessarily all such advantages may be achieved in accordance with any particular embodiments of the disclosure. Thus, the disclosure may be embodied or carried out in a manner that achieves or optimizes one advantage or group of advantages as taught herein without necessarily achieving other advantages as may be taught or suggested herein.

Additionally, although embodiments of the disclosure have been described in detail, certain variations and modifications will be apparent to those skilled in the art, including embodiments that do not provide all the features and benefits described herein. It will be understood by those skilled in the art that the present disclosure extends beyond the specifically disclosed embodiments to other alternative or additional embodiments and/or uses and obvious modifications and equivalents thereof. Moreover, while a number of variations have been shown and described in varying detail, other modifications, which are within the scope of the present disclosure, will be readily apparent to those of skill in the art based upon this disclosure. It is also contemplated that various combinations or sub-combinations of the specific features and aspects of the embodiments may be made and still fall within the scope of the present disclosure. Accordingly, it should be understood that various features and aspects of the disclosed embodiments can be combined with or substituted for one another in order to form varying modes of the present disclosure. Thus, it is intended that the scope of the present disclosure herein disclosed should not be limited by the particular disclosed embodiments described herein.

As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

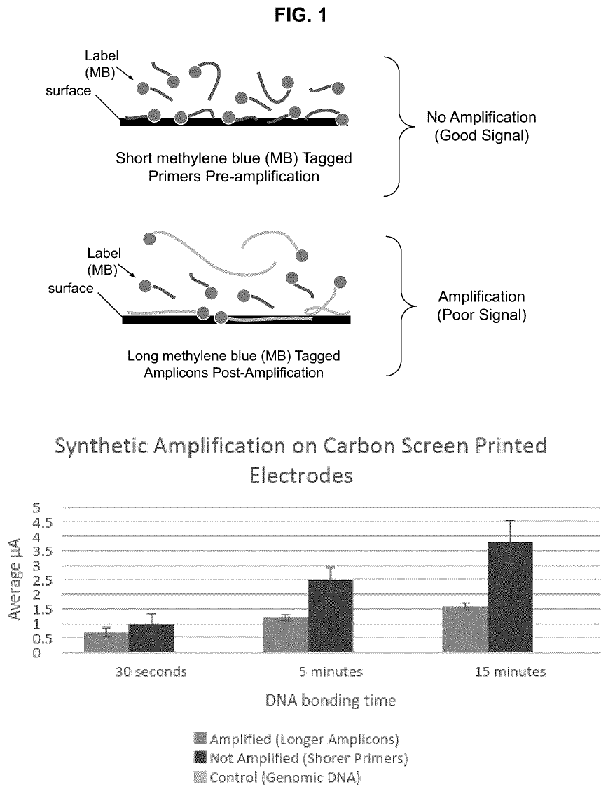

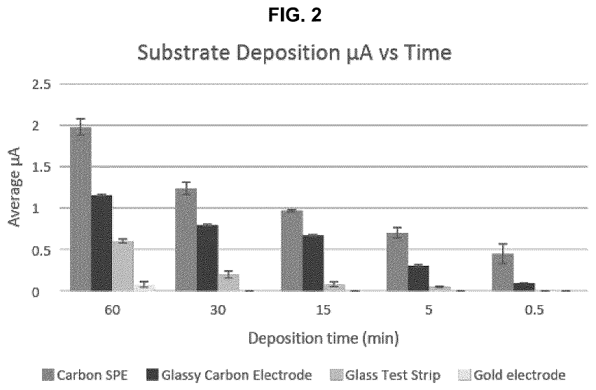

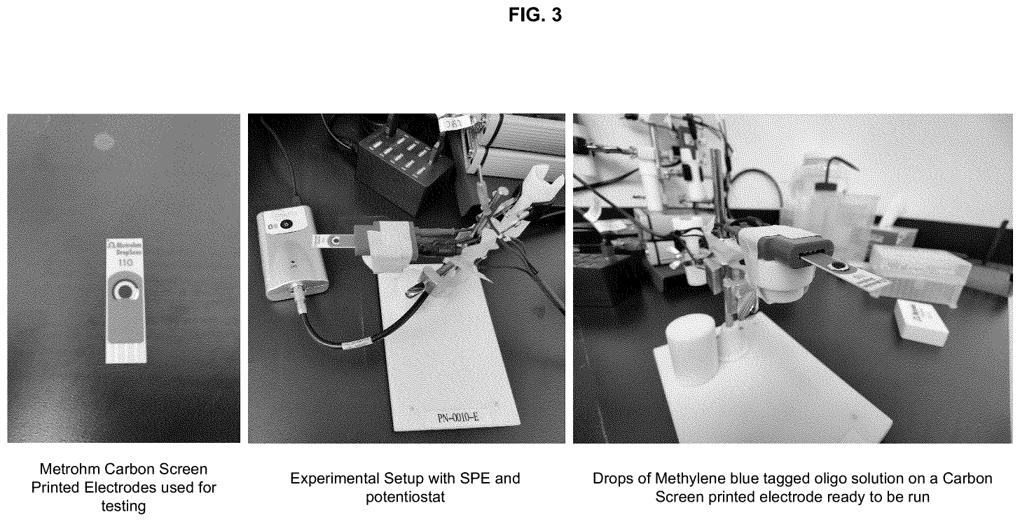

Referring now to FIGS. 1-3, the present invention features methods, systems, and compositions for probeless detection of oligonucleotides. The methods, systems, and compositions can be used for a variety of applications, such as but not limited to determining if a particular sample contains amplicons derived from a set of tagged oligonucleotide primers. For example, the methods, systems, and compositions can be used to compare the amount of labeled primers in a sample prior to amplification to the amount of labeled primers in a sample after amplification to determine if amplification occurred.

### Sensor

A sensor is constructed comprising an electrode surface and tagged DNA or RNA oligonucleotide primers bound thereon. In some embodiments, the electrode surface comprises a carbon screen printed electrode (SPE), a glassy carbon electrode, a glass strip, or a gold electrode.

The present invention features a system comprising a surface and a plurality of oligonucleotides adhered to the surface. At least a portion of the oligonucleotides are labeled with an electrochemical label. In some embodiments, the system is for detecting target oligonucleotides. In other embodiments, the system is for detecting the amplification of target oligonucleotides.

In some embodiments, the target oligonucleotides comprise DNA, RNA, or a combination thereof. In some embodiments, the target oligonucleotides are single-stranded. In other embodiments, the target oligonucleotides are double-stranded.

In some embodiments, the surface comprises an electrode surface. In some embodiments, the surface comprises a carbon screen printed electrode, platinum, a glassy carbon electrode, a glass strip, a gold electrode, a carbon screen printed electrode modified with carbon nanotubes, a carbon screen printed electrode modified with a metal nanoparticle, platinum modified with carbon nanotubes, platinum modified with a metal nanoparticle, a glassy carbon electrode modified with carbon nanotubes, a glassy carbon electrode modified with a metal nanoparticle, a glass strip modified with carbon nanotubes, a glass strip modified with a metal nanoparticle, a gold electrode modified with carbon nanotubes, or a gold electrode modified with a metal nanoparticle.

In some embodiments, the metal nanoparticle comprises gold, silver, iron, cobalt, platinum, carbon, or copper.

In contrast to the techniques used in the art, the present invention does not use probes bound to the surface, and the targets are measured without probes. Thus, the surface is free of single-stranded probe compositions, e.g., single-stranded oligonucleotides attached to the surface via a free thiol moiety at a 3′ end, single-stranded oligonucleotides complementary to the oligonucleotides labeled with the electrochemical label, etc.

In some embodiments, the electrochemical label (or tag) comprises methylene blue, methylene violet, ruthenium hexamine, ferrocene, anthraquinone, an anthraquinone derivative, a metal nanoparticle (e.g., a metal nanoparticle comprising gold, silver, iron, cobalt, platinum, carbon, copper, or other appropriate metal (including core-shell variations)), the like, or a combination thereof.

In some embodiments, the target oligonucleotides comprise single-stranded DNA or RNA. In some embodiments, the length of the oligonucleotides is from 10 to 20 nucleotides. In some embodiments, the length of the oligonucleotides is from 10 to 30 nucleotides. In some embodiments, the length of the oligonucleotides is from 20 to 50 nucleotides. In some embodiments, the length of the oligonucleotides is from 10 to 50 nucleotides. In some embodiments, the length of the oligonucleotides is from 30 to 55 nucleotides. In some embodiments, the length of the oligonucleotides is from 75 to 100 nucleotides. In some embodiments, the length of the oligonucleotides is from 80 to 100 nucleotides. In some embodiments, the length of the oligonucleotides is from 90 to 110 nucleotides. In some embodiments, the length of the oligonucleotides is from 80 to 120 nucleotides. The present invention is not limited to the aforementioned lengths of primers and amplicons.

In some embodiments, when the target oligonucleotides are short (e.g., less than 50 nucleotides), the electrochemical label is able to interact with the surface of the sensor, and a larger current peak is seen. In some embodiments, when the target oligonucleotides are long (e.g., greater than 50), the electrochemical label interacts with the surface of the sensor less, thus decreasing the measured current. See FIG. 1 for an example. The present invention is not limited to the term “short,” referring to 50 or fewer nucleotides, and the term “long,” referring to greater than 50 nucleotides. The definitions of “short” and “long” depend on the difference between amplified and non-amplified oligonucleotides; e.g., the terms “short” and “long” are defined as a number relative to the amplified and non-amplified oligonucleotides.

In some embodiments, the present invention features a system comprising a carbon screen printed electrode (SPE) surface and a plurality of single-stranded DNA oligonucleotides adhered to the carbon SPE surface. At least a portion of the single-stranded DNA oligonucleotides are labeled with an electrochemical label selected from methylene blue, methylene violet, ruthenium hexamine, ferrocene, anthraquinone, an anthraquinone derivative, a metal nanoparticle, or a combination thereof.

The aforementioned systems may further comprise a potentiostat for detecting oligonucleotides labeled with the electrochemical label adhered to the surface. For example, the potentiostat may detect single-stranded DNA oligonucleotides labeled with the electrochemical label adhered to the surface.

The present invention may also feature a kit, e.g., a kit comprising a surface for adhering a plurality of oligonucleotides (e.g., ssDNA) and an electrochemical label for labeling the plurality of oligonucleotides (e.g., target oligonucleotides). In some embodiments, the kit comprises a surface for adhering a plurality of oligonucleotides (e.g., ssDNA) and one or more primers. The primers that are different and are specific for different target oligonucleotides will each have a unique electrochemical label. Multiplexing is possible when each electrochemical label is different.

### Methods of Preparing Sensors Herein

The present invention may also feature a method of preparing a surface for oligonucleotide detection. The method may comprise incubating a surface with a plurality of oligonucleotides for a period of time, wherein at least a portion of the oligonucleotides are labeled with an electrochemical label.

In some embodiments, the surface is incubated with a solution of the plurality of oligonucleotides at a ratio of at least 20 μL of solution per 10 mm² of the surface. In other embodiments, the surface is incubated with a solution of the plurality of oligonucleotides at a ratio of at least 15 μL of solution per 10 mm² of the surface. In other embodiments, the surface is incubated with a solution of the plurality of oligonucleotides at a ratio of at least 10 μL of solution per 10 mm² of the surface. In other embodiments, the surface is incubated with a solution of the plurality of oligonucleotides at a ratio of at least 5 μL of solution per 10 mm2 of the surface. In other embodiments, the surface is incubated with a solution of the plurality of oligonucleotides at a ratio of at least 1 μL of solution per 10 mm² of the surface.

In other embodiments, the surface is incubated with a solution of the plurality of oligonucleotides at a ratio of about 1 μL to 20 μL, or about 1 μL to 15 μL, or about 1 μL to 10 μL, or about 1 μL to 5 μL, or about 5 μL to 20 μL, or about 5 μL to 15 μL, or about 5 μL to 10 μL, or about 10 μL to 20 μL, or about 10 μL to 15 μL, or about 15 μL to 20 μL of solution per 10 mm² of the surface.

In some embodiments, the plurality of oligonucleotides is at a concentration of at least 10 nM. In some embodiments, the plurality of oligonucleotides is at a concentration of at least 15 nM. In some embodiments, the plurality of oligonucleotides is at a concentration of at least 25 nM. In some embodiments, the plurality of oligonucleotides is at a concentration of at least 40 nM. In some embodiments, the plurality of oligonucleotides is at a concentration of at least 50 nM. In some embodiments, the plurality of oligonucleotides is at a concentration of at least 75 nM. In some embodiments, the plurality of oligonucleotides is at a concentration of at least 100 nM.

In other embodiments, the plurality of oligonucleotides is at a concentration of about 10 nM to 100 nM, or about 10 nM to 75 nM, or about 10 nM to 50 nM, or about 10 nM to 40 nM, or about 10 nM to 25 nM, or about 10 nM to 15 nM, or about 15 nM to 100 nM, or about 15 nM to 75 nM, or about 15 nM to 50 nM, or about 15 nM to 40 nM, or about 15 nM to 25 nM. In further embodiments, the plurality of oligonucleotides is at a concentration of about 25 nM to 100 nM, or about 25 nM to 75 nM, or about 25 nM to 50 nM, or about 25 nM to 40 nM, or about 40 nM to 100 nM, or about 40 nM to 75 nM, or about 40 nM to 50 nM, or about 50 nM to 100 nM, or about 50 nM to 75 nM, or about 75 nM to 100 nM.

In some embodiments, the sensors may be created by depositing a volume (e.g., 15 μL to 100 μL) of labeled oligonucleotides onto the surface and allowing it to adhere for a period of time.

In some embodiments, the volume of labeled oligonucleotides deposited onto the surface is about 10 μL to 100 μL, or about 10 μL to 75 μL, or about 10 μL to 50 μL, or about 10 μL to 20 μL, or about 10 μL to 15 μL. In some embodiments, the volume of labeled oligonucleotides deposited onto the surface is about 15 μL to 100 μL, or about 15 μL to 75 μL, or about 15 μL to 50 μL, or about 15 μL to 20 μL. In some embodiments, the volume of labeled oligonucleotides deposited onto the surface is about 50 μL to 100 μL, or about 50 μL to 75 μL, or about 75 μL to 100 μL. In some embodiments, the volume of labeled oligonucleotides deposited onto the surface is more than 100 μL.

In some embodiments, the period of time is at least 30 seconds. In some embodiments, the period of time is about 30 seconds to 60 seconds. In some embodiments, the period of time is about 1 minute to 10 minutes. In some embodiments, the period of time is about 10 minutes to 30 minutes. In some embodiments, the period of time is about 30 minutes to 60 minutes. In some embodiments, the period of time is about 60 minutes to 120 minutes.

In other embodiments, the period of time is about 30 seconds to 120 minutes, or about 30 seconds to 60 minutes, or about 30 seconds to 30 minutes, or about 30 seconds to 20 minutes, or about 30 seconds to 10 minutes, or about 30 seconds to 5 minutes, or about 5 minutes to 120 minutes, or about 5 minutes to 60 minutes, or about 5 minutes to 30 minutes, or about 5 minutes to 20 minutes or about 5 minutes to 10 minutes. In other embodiments, the period of time is about 10 minutes to 120 minutes, or about 10 minutes to 60 minutes, or about 10 minutes to 30 minutes, or about 10 minutes to 20 minutes, or about 30 minutes to 120 minutes, or about 30 minutes to 60 minutes.

### Methods of Use

The present invention may also feature a method of detecting an oligonucleotide. The method may comprise (a) incubating a surface with a volume of a solution comprising a plurality of oligonucleotides for a period of time, wherein at least a portion of the oligonucleotides are labeled with an electrochemical label and (b) detecting an amount of oligonucleotides adhered to the surface using a potentiostatic system which detects the electrochemical labels of the oligonucleotides.

The present invention may further feature a method of detecting amplification of a target oligonucleotide. The method may comprise (a) incubating a first surface with volume of a first solution comprising a plurality of single-stranded primers specific for the target oligonucleotide for a period of time, wherein at least a portion of the single-stranded primers are labeled with an electrochemical label and (b) detecting an amount of single stranded primers in the first solution adhered to the first surface using a potentiostatic system, which detects the electrochemical labels of the single-stranded primers. The method may further comprise incubating a second surface with a volume of a second solution for a period of time, and detecting an amount of single-stranded primers in the second solution adhered to the second surface using a potentiostatic system, which detects the electrochemical labels of the single stranded primers. In some embodiments, the second surface has the same composition as the first surface. The second solution may comprise one or both of: (i) a plurality of single-stranded primers specific for the target oligonucleotide, wherein at least a portion of the single-stranded primers are labeled with an electrochemical label, or (ii) target oligonucleotide amplicons derived from the plurality of single-stranded primers specific for the target oligonucleotide. Lastly, the method may comprise comparing the amount of single-stranded primers in the first solution adhered to the first surface with the amount of single-stranded primers in the second solution adhered to the second surface, wherein if the amount of single-stranded primers in the second solution adhered to the second surface is less than the amount of single-stranded primers in the first solution adhered to the first surface, then the second solution comprises amplified target oligonucleotides.

In some embodiments, the potentiostatic system is a square wave voltammetry system. The potentiostatic system may be a cyclic voltammetry system, a chronoamperometry system, or an electrochemical impedance spectroscopy system.

Square wave voltammetry may be used to measure the reaction between the electrochemical label and the surface of the sensor. In some embodiments, the electrochemical label transfers electrons to and from the surface, creating a voltage-dependent current that can be measured with a potentiostat. The potentiostat is the equipment that records the current that is generated by the voltage at the electrodes (i.e., what is plotted by a computer) and controls the signals generated/used to perform square wave voltammetry.

In some embodiments, the setup comprises a potentiostat and one or more electrodes. In other embodiments, the setup comprises a potentiostat and two or more electrodes. In further embodiments, the setup comprises a potentiostat and one electrode, or two electrodes, or three electrodes, or four electrodes, or more electrodes. In some embodiments, the setup comprises a potentiostat, a sensor as described herein (i.e., a working electrode), and a reference electrode. In other embodiments, the setup comprises a potentiostat, a sensor as described herein (i.e., a working electrode), a reference electrode, and a counter electrode.

As used herein, a “reference electrode” refers to an electrode that has a stable and well-known electrode potential. Non-limiting examples of reference electrodes may include but are not limited to stable reference electrodes such as a calomel electrode or a quasi-reference electrode like a chloridized silver wire. In preferred embodiments, the reference electrode comprises a 3M KCl/AgCl/Ag reference electrode. As used herein, a “counter electrode” or an “auxiliary electrode” may be used interchangeably and refers to an electrode that is used to close the current circuit in the electrochemical cell and does not participate in the electrochemical reaction. Non-limiting examples of counter electrodes include but are not limited to an unmodified Platinum electrode or an unmodified gold electrode.

Square wave voltammetry may be run with the tagged oligonucleotide solution still on the surface of the electrode. The tagged oligonucleotides result in a signal that changes strength depending on the length and/or concentration of tagged oligonucleotides. The deposition time (the period of time the oligonucleotides are deposited onto the surface) can also affect the signal strength.

The present invention also includes the use of the sensor with double stranded DNA. In some embodiments, the surface is pre-treated with an adhesion promoting composition or layer to help the double stranded DNA adhere better to the surface.

### EXAMPLE

The following is a non-limiting example of the present invention. It is to be understood that said example is not intended to limit the present invention in any way. Equivalents or substitutes are within the scope of the present invention.

Step 1) A 30 mer methylene blue tagged “synthetic primer” oligo and a 108 mer methylene blue tagged “synthetic amplicon” oligo are synthesized. In some embodiments, amplicons are derived from amplification techniques. A non-limiting example of an amplification technique includes single-stranded isothermal amplification.

Step 2) Both the Primer and Amplicon solutions are diluted to a 1000 nM concentration with 1×PBS.

Step 3) A Metrohm Carbon Screen printed electrode (SPE) is placed into a Palmsens SPE connector and attached to a Palmsens Emstat3 Blue or similar potentiostat.

Step 4) 40 μL of the 1000 nM Primer solution is placed directly onto the working electrode of the SPE and allowed to deposit for 15 minutes.

Step 5) After the 15 minute deposition time, square wave voltammetry is run with the following settings; Conditioning potential: −0.45 μA. Conditioning time: 5 seconds. Disposition potential: 0. Disposition time: 0. Equilibration time: 2 seconds. Beginning potential: −0.45 μA. End potential: 0 μA. Step Potential: 0.001 μA. Pulse Amplitude: 0.025 μA. Frequency: 60. Measure forward reverse: false.

Step 6) Steps 3-5 are repeated but with the 1000 nM amplicon solution instead of the primer solution.

Step 7) Signal amplitudes are compared.

### EMBODIMENTS

The following embodiments are intended to be illustrative only and not to be limiting in any way.

Embodiment 1: A system comprising: a) a surface; and b) a plurality of oligonucleotides adhered to the surface, wherein at least a portion of the oligonucleotides are labeled with an electrochemical label.

Embodiment 2: The system of embodiment 1, wherein the system is for oligonucleotide detection.

Embodiment 3: The system of embodiment 1 or embodiment 2, wherein the system is for detecting amplification of a target oligonucleotide.

Embodiment 4: The system of any one of embodiments 1-3, wherein the oligonucleotides comprise DNA or RNA.

Embodiment 5: The system of any one of embodiments 1-4, wherein the oligonucleotides are single-stranded.

Embodiment 6: The system of any one of embodiments 1-4, wherein the oligonucleotides are double-stranded.

Embodiment 7: The system of any one of embodiments 1-6, wherein the surface comprises a carbon screen printed electrode, platinum, a glassy carbon electrode, a glass strip, a gold electrode, a carbon screen printed electrode modified with carbon nanotubes, a carbon screen printed electrode modified with a metal nanoparticle, platinum modified with carbon nanotubes, platinum modified with a metal nanoparticle, a glassy carbon electrode modified with carbon nanotubes, a glassy carbon electrode modified with a metal nanoparticle, a glass strip modified with carbon nanotubes, a glass strip modified with a metal nanoparticle, a gold electrode modified with carbon nanotubes, or a gold electrode modified with a metal nanoparticle.

Embodiment 8: The system of embodiment 7, wherein the metal nanoparticle comprises gold, silver, iron, cobalt, platinum, carbon, or copper.

Embodiment 9: The system of any one of embodiments 1-8, wherein the electrochemical label comprises methylene blue, methylene violet, ruthenium hexamine, ferrocene, anthraquinone, an anthraquinone derivative, a metal nanoparticle, or a combination thereof.

Embodiment 10: The system of embodiment 9, wherein the metal nanoparticle comprises gold, silver, iron, cobalt, platinum, carbon, or copper.

Embodiment 11: The system of any one of embodiments 1-10, wherein the surface is free of a single-stranded probe composition comprising single-stranded oligonucleotides complementary to the oligonucleotides labeled with the electrochemical label.

Embodiment 12: The system of any one of embodiments 1-11 further comprising a potentiostat for detecting oligonucleotides labeled with the electrochemical label adhered to the surface.

Embodiment 13: A system comprising: a) a carbon screen printed electrode (SPE) surface; and b) a plurality of single-stranded DNA oligonucleotides adhered to the carbon SPE surface, wherein at least a portion of the single-stranded DNA oligonucleotides are labeled with an electrochemical label selected from methylene blue, methylene violet, ruthenium hexamine, ferrocene, anthraquinone, an anthraquinone derivative, a metal nanoparticle, or a combination thereof.

Embodiment 14: The system of embodiment 13, wherein the system is for oligonucleotide detection.

Embodiment 15: The system of embodiment 13 or embodiment 14, wherein the system is for detecting amplification of a target oligonucleotide.

Embodiment 16: The system of any one of embodiments 13-15, wherein the metal nanoparticle comprises gold, silver, iron, cobalt, platinum, carbon, or copper.

Embodiment 17: The system of any one of embodiments 13-16, wherein the surface is free of a single-stranded probe composition comprising single-stranded oligonucleotides complementary to the oligonucleotides labeled with the electrochemical label.

Embodiment 18: The system of any one of embodiments 13-17 further comprising a potentiostat for detecting single-stranded DNA oligonucleotides labeled with the electrochemical label adhered to the surface.

Embodiment 19: A kit comprising: a) a surface for adhering a plurality of oligonucleotides; and b) an electrochemical label for labeling the plurality of oligonucleotides.

Embodiment 20: The kit of embodiment 19, wherein the surface is selected from: a carbon screen printed electrode, platinum, a glassy carbon electrode, a glass strip, a gold electrode, a carbon screen printed electrode modified with carbon nanotubes, a carbon screen printed electrode modified with a metal nanoparticle, platinum modified with carbon nanotubes, platinum modified with a metal nanoparticle, a glassy carbon electrode modified with carbon nanotubes, a glassy carbon electrode modified with a metal nanoparticle, a glass strip modified with carbon nanotubes, a glass strip modified with a metal nanoparticle, a gold electrode modified with carbon nanotubes, or a gold electrode modified with a metal nanoparticle.

Embodiment 21: The kit of embodiment 20, wherein the metal nanoparticle comprises gold, silver, iron, cobalt, platinum, carbon, or copper.

Embodiment 22: The kit of any one of embodiments 19-21, wherein the electrochemical label is selected from methylene blue, methylene violet, ruthenium hexamine, anthraquinone, an anthraquinone derivative, a metal nanoparticle, or a combination thereof.

Embodiment 23: The kit of embodiment 22, wherein the metal nanoparticle comprises gold, silver, iron, cobalt, platinum, carbon, or copper.

Embodiment 24: A method of preparing a surface for oligonucleotide detection, said method comprising: incubating a surface with a plurality of oligonucleotides for a period of time, wherein at least a portion of the oligonucleotides are labeled with an electrochemical label.

Embodiment 25: The method of embodiment 24, wherein the surface is incubated with a solution of the plurality of oligonucleotides at a ratio of at least 20 μL of solution per 10 mm² of the surface.

Embodiment 26: The method of embodiment 24, wherein the surface is incubated with a solution of the plurality of oligonucleotides at a ratio of at least 10 μL of solution per 10 mm² of the surface.

Embodiment 27: The method of any one of embodiments 24-26, wherein the plurality of oligonucleotides is at a concentration of at least 10 nM.

Embodiment 28: The method of any one of embodiments 24-26, wherein the plurality of oligonucleotides is at a concentration of at least 40 nM.

Embodiment 29: The method of any one of embodiments 24-26, wherein the plurality of oligonucleotides is at a concentration of at least 100 nM.

Embodiment 30: The method of any one of embodiments 24-29, wherein the period of time is at least 30 seconds.

Embodiment 31: The method of any one of embodiments 24-29, wherein the period of time is from 30-60 seconds.

Embodiment 32: The method of any one of embodiments 24-29, wherein the period of time is from 1 to 10 minutes.

Embodiment 33: The method of any one of embodiments 24-29, wherein the period of time is from 10 to 30 minutes.

Embodiment 34: The method of any one of embodiments 24-29, wherein the period of time is from 30 to 60 minutes.

Embodiment 35: The method of any one of embodiments 24-29, wherein the period of time is from 60 to 120 minutes.

Embodiment 36: The method of any one of embodiments 24-35, wherein the surface is selected from: a carbon screen printed electrode, platinum, a glassy carbon electrode, a glass strip, a gold electrode, a carbon screen printed electrode modified with carbon nanotubes, a carbon screen printed electrode modified with a metal nanoparticle, platinum modified with carbon nanotubes, platinum modified with a metal nanoparticle, a glassy carbon electrode modified with carbon nanotubes, a glassy carbon electrode modified with a metal nanoparticle, a glass strip modified with carbon nanotubes, a glass strip modified with a metal nanoparticle, a gold electrode modified with carbon nanotubes, or a gold electrode modified with a metal nanoparticle.

Embodiment 37: The method of embodiment 36, wherein the metal nanoparticle comprises gold, silver, iron, cobalt, platinum, carbon, or copper.

Embodiment 38: The method of any one of embodiments 24-37, wherein the electrochemical label is selected from methylene blue, methylene violet, ruthenium hexamine, ferrocene, anthraquinone, an anthraquinone derivative, a metal nanoparticle, or a combination thereof.

Embodiment 39: The method of embodiment 38, wherein the metal nanoparticle comprises gold, silver, iron, cobalt, platinum, carbon, or copper.

Embodiment 40: A method of detecting an oligonucleotide, the method comprising: a) incubating a surface with a volume of a solution comprising a plurality of oligonucleotides for a period of time, wherein at least a portion of the oligonucleotides are labeled with an electrochemical label; and b) detecting an amount of oligonucleotides adhered to the surface using a potentiostatic system which detects the electrochemical labels of the oligonucleotides.

Embodiment 41: The method of embodiment 40, wherein the potentiostatic system is a square wave voltammetry system.

Embodiment 42: The method embodiment 40 or embodiment 41, wherein the surface is selected from: a carbon screen printed electrode, platinum, a glassy carbon electrode, a glass strip, a gold electrode, a carbon screen printed electrode modified with carbon nanotubes, a carbon screen printed electrode modified with a metal nanoparticle, platinum modified with carbon nanotubes, platinum modified with a metal nanoparticle, a glassy carbon electrode modified with carbon nanotubes, a glassy carbon electrode modified with a metal nanoparticle, a glass strip modified with carbon nanotubes, a glass strip modified with a metal nanoparticle, a gold electrode modified with carbon nanotubes, or a gold electrode modified with a metal nanoparticle.

Embodiment 43: The method of embodiment 42, wherein the metal nanoparticle comprises gold, silver, iron, cobalt, platinum, carbon, or copper.

Embodiment 44: The method of any one of embodiments 40-43, wherein the electrochemical label is selected from methylene blue, methylene violet, ruthenium hexamine, ferrocene, anthraquinone, an anthraquinone derivative, a metal nanoparticle, or a combination thereof.

Embodiment 45: The system of embodiment 44, wherein the metal nanoparticle comprises gold, silver, iron, cobalt, platinum, carbon, or copper.

Embodiment 46: A method of detecting amplification of a target oligonucleotide, the method comprising: a) incubating a first surface with volume of a first solution comprising a plurality of single-stranded primers specific for the target oligonucleotide for a period of time, wherein at least a portion of the single-stranded primers are labeled with an electrochemical label; b) detecting an amount of single stranded primers in the first solution adhered to the first surface using a potentiostatic system, which detects the electrochemical labels of the single-stranded primers; c) incubating a second surface with volume of a second solution for a period of time, wherein the second surface has the same composition as the first surface, wherein the second solution comprises one or both of: (i) a plurality of single-stranded primers specific for the target oligonucleotide, wherein at least a portion of the single-stranded primers are labeled with an electrochemical label, or (ii) target oligonucleotide amplicons derived from the plurality of single stranded primers specific for the target oligonucleotide; d) detecting an amount of single stranded primers in the second solution adhered to the second surface using a potentiostatic system, which detects the electrochemical labels of the single stranded primers; and e) comparing the amount of single stranded primers in the first solution adhered to the first surface with the amount of single stranded primers in the second solution adhered to the second surface, wherein if the amount of single stranded primers in the second solution adhered to the second surface is less than the amount of single stranded primers in the first solution adhered to the first surface, then the second solution comprises amplified target oligonucleotides.

Embodiment 47: The method of embodiment 46, wherein the potentiostatic system is a square wave voltammetry system.

Embodiment 48: The method of embodiment 46, wherein the potentiostatic system is a cyclic voltammetry system, a chronoamperometry system, or an electrochemical impedance spectroscopy system.

Embodiment 49: The method of any of embodiments 46-48, wherein the single-stranded primers are from 10-50 nucleotides in length.

Embodiment 50: The method of any of embodiments 46-49, wherein the amplicons are from 80 to 120 nucleotides in length.

Embodiment 51: The method of any one of embodiments 46-50, wherein the first surface is selected from: a carbon screen printed electrode, platinum, a glassy carbon electrode, a glass strip, a gold electrode, a carbon screen printed electrode modified with carbon nanotubes, a carbon screen printed electrode modified with a metal nanoparticle, platinum modified with carbon nanotubes, platinum modified with a metal nanoparticle, a glassy carbon electrode modified with carbon nanotubes, a glassy carbon electrode modified with a metal nanoparticle, a glass strip modified with carbon nanotubes, a glass strip modified with a metal nanoparticle, a gold electrode modified with carbon nanotubes, or a gold electrode modified with a metal nanoparticle.

Embodiment 52: The method of embodiment 51, wherein the metal nanoparticle comprises gold, silver, iron, cobalt, platinum, carbon, or copper.

Embodiment 53: The method of any one of embodiments 46-52, wherein the electrochemical label is selected from methylene blue, methylene violet, ruthenium hexamine, ferrocene, anthraquinone, an anthraquinone derivative, a metal nanoparticle, or a combination thereof.

Embodiment 54: The method of embodiment 53, wherein the metal nanoparticle comprises gold, silver, iron, cobalt, platinum, carbon, or copper.

Embodiment 55: A method of detecting amplification of a target oligonucleotide, the method comprising: a) incubating a first surface with volume of a first solution comprising a plurality of single stranded, electrochemically-labeled primers specific for the target oligonucleotide for a period of time; b) detecting an amount of single stranded, electrochemically-labeled primers in the first solution adhered to the first surface using a potentiostatic system, which detects electrochemical labels of the single stranded, electrochemically-labeled primers; c) incubating a second surface with volume of a second solution for a period of time, wherein the second surface has the same composition as the first surface, wherein the second solution comprises one or both of: (i) a plurality of single-stranded, electrochemically-labeled primers specific for the target oligonucleotide, or (ii) target oligonucleotide amplicons formed from the plurality of single-stranded, electrochemically-labeled primers specific for the target oligonucleotide; d) detecting an amount of single stranded, electrochemically-labeled primers in the second solution adhered to the second surface using a potentiostatic system, which detects electrochemical labels of the single stranded, electrochemically-labeled primers; and e) comparing the amount of single stranded, electrochemically-labeled primers in the first solution adhered to the first surface with the amount of single stranded, electrochemically-labeled primers in the second solution adhered to the second surface; wherein if the amount of single stranded, electrochemically-labeled primers adhered to the second surface is less than the amount of single stranded, electrochemically-labeled primers adhered to the first surface, then the second solution comprises amplified target oligonucleotides.

Embodiment 56: The method of embodiment 55, wherein the potentiostatic system is a square wave voltammetry system.

Embodiment 57: The method of embodiment 56, wherein the potentiostatic system is a cyclic voltammetry system, a chronoamperometry system, or an electrochemical impedance spectroscopy system.

Embodiment 58: The method of embodiment 56 or embodiment 57, wherein the single-stranded primers are from 10-50 nucleotides in length.

Embodiment 59: The method of any one of embodiments 56-58, wherein the amplicons are from 80 to 120 nucleotides in length.

Embodiment 60: The method of any one of embodiments 56-59, wherein the first surface is selected from: a carbon screen printed electrode, platinum, a glassy carbon electrode, a glass strip, a gold electrode, a carbon screen printed electrode modified with carbon nanotubes, a carbon screen printed electrode modified with a metal nanoparticle, platinum modified with carbon nanotubes, platinum modified with a metal nanoparticle, a glassy carbon electrode modified with carbon nanotubes, a glassy carbon electrode modified with a metal nanoparticle, a glass strip modified with carbon nanotubes, a glass strip modified with a metal nanoparticle, a gold electrode modified with carbon nanotubes, or a gold electrode modified with a metal nanoparticle.

Embodiment 61: The method of embodiment 60, wherein the metal nanoparticle comprises gold, silver, iron, cobalt, platinum, carbon, or copper.

Embodiment 62: The method of any one of embodiments 56-61, wherein the electrochemical label is selected from methylene blue, methylene violet, ruthenium hexamine, ferrocene, anthraquinone, an anthraquinone derivative, a metal nanoparticle, or a combination thereof.

Embodiment 63: The method of embodiment 62, wherein the metal nanoparticle comprises gold, silver, iron, cobalt, platinum, carbon, or copper.

Embodiment 64: A system comprising: a) a surface selected from: a carbon screen printed electrode, platinum, a glassy carbon electrode, a glass strip, a gold electrode, a carbon screen printed electrode modified with carbon nanotubes, a carbon screen printed electrode modified with a metal nanoparticle, platinum modified with carbon nanotubes, platinum modified with a metal nanoparticle, a glassy carbon electrode modified with carbon nanotubes, a glassy carbon electrode modified with a metal nanoparticle, a glass strip modified with carbon nanotubes, a glass strip modified with a metal nanoparticle, a gold electrode modified with carbon nanotubes, or a gold electrode modified with a metal nanoparticle; and b) a plurality of single-stranded DNA oligonucleotides adhered to the carbon SPE surface via Van der Waals forces, wherein at least a portion of the single-stranded DNA oligonucleotides are labeled with an electrochemical label.

Embodiment 65: The system of embodiment 64, wherein the single-stranded DNA oligonucleotides are single-stranded primers, or are single-stranded DNA amplicons comprising single-stranded primers.

Embodiment 66: The system of embodiment 64 or embodiment 65, wherein the single-stranded primers are from 10-50 nucleotides in length.

Embodiment 67: The system of any one of embodiments 64-66, wherein the amplicons are from 80 to 120 nucleotides in length.

Embodiment 68: The system of any one of embodiments 64-67, wherein the metal nanoparticle comprises gold, silver, iron, cobalt, platinum, carbon, or copper.

Embodiment 69: The system of any one of embodiments 64-68, wherein the electrochemical label is selected from methylene blue, methylene violet, ruthenium hexamine, ferrocene, anthraquinone, an anthraquinone derivative, a metal nanoparticle, or a combination thereof.

Embodiment 70: A system comprising: a) a carbon screen printed electrode (SPE); and b) a plurality of single-stranded DNA oligonucleotides adhered to the carbon SPE surface via Van der Waals forces, wherein at least a portion of the single-stranded DNA oligonucleotides are labeled with an electrochemical label.

Embodiment 71: The system of embodiment 70, wherein the single-stranded DNA oligonucleotides are single-stranded primers or are single-stranded DNA amplicons comprising single-stranded primers.

Embodiment 72: The system of embodiment 70 or embodiment 71, wherein the single-stranded primers are from 10-50 nucleotides in length.

Embodiment 73: The system of any one of embodiments 70-72, wherein the amplicons are from 80 to 120 nucleotides in length.

Embodiment 74: The system of any one of embodiments 70-73, wherein the electrochemical label is selected from methylene blue, methylene violet, ruthenium hexamine, ferrocene, anthraquinone, an anthraquinone derivative, a metal nanoparticle, or a combination thereof.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. A method of detecting amplification of a target oligonucleotide, the method comprising:
a) incubating a first surface with a volume of a first solution comprising a plurality of single stranded, electrochemically-labeled primers specific for the target oligonucleotide for a period of time;
b) detecting an amount of single stranded, electrochemically-labeled primers in the first solution adhered to the first surface using a potentiostatic system, which detects electrochemical labels of the single stranded, electrochemically-labeled primers;
c) incubating a second surface with a volume of a second solution for a period of time, wherein the second surface has the same composition as the first surface, wherein the second solution comprises one or both of: (i) a plurality of single-stranded, electrochemically-labeled primers specific for the target oligonucleotide, or (ii) target oligonucleotide amplicons formed from the plurality of single-stranded, electrochemically-labeled primers specific for the target oligonucleotide;
d) detecting an amount of single stranded, electrochemically-labeled primers in the second solution adhered to the second surface using a potentiostatic system, which detects electrochemical labels of the single stranded, electrochemically-labeled primers; and
e) comparing the amount of single stranded, electrochemically-labeled primers in the first solution adhered to the first surface with the amount of single stranded, electrochemically-labeled primers in the second solution adhered to the second surface;
wherein if the amount of single stranded, electrochemically-labeled primers adhered to the second surface is less than the amount of single stranded, electrochemically-labeled primers adhered to the first surface, then the second solution comprises amplified target oligonucleotides.

2. The method of claim 1, wherein the potentiostatic system is a square wave voltammetry system.

3. The method of claim 2, wherein the potentiostatic system is a cyclic voltammetry system, a chronoamperometry system, or an electrochemical impedance spectroscopy system.

4. The method of claim 1, wherein the single-stranded primers are from 10-50 nucleotides in length.

5. The method of claim 1, wherein the amplicons are from 80 to 120 nucleotides in length.

6. The method of claim 1, wherein the first surface is selected from: a carbon screen printed electrode, platinum, a glassy carbon electrode, a glass strip, a gold electrode, a carbon screen printed electrode modified with carbon nanotubes, a carbon screen printed electrode modified with a metal nanoparticle, platinum modified with carbon nanotubes, platinum modified with a metal nanoparticle, a glassy carbon electrode modified with carbon nanotubes, a glassy carbon electrode modified with a metal nanoparticle, a glass strip modified with carbon nanotubes, a glass strip modified with a metal nanoparticle, a gold electrode modified with carbon nanotubes, or a gold electrode modified with a metal nanoparticle.

7. The method of claim 6, wherein the metal nanoparticle comprises gold, silver, iron, cobalt, platinum, carbon, or copper.

8. The method of claim 1, wherein the electrochemical label is selected from methylene blue, methylene violet, ruthenium hexamine, ferrocene, anthraquinone, an anthraquinone derivative, a metal nanoparticle, or a combination thereof.

9. The method of claim 8, wherein the metal nanoparticle comprises gold, silver, iron, cobalt, platinum, carbon, or copper.

10. A system comprising:
a) a surface selected from: a carbon screen printed electrode, platinum, a glassy carbon electrode, a glass strip, a gold electrode, a carbon screen printed electrode modified with carbon nanotubes, a carbon screen printed electrode modified with a metal nanoparticle, platinum modified with carbon nanotubes, platinum modified with a metal nanoparticle, a glassy carbon electrode modified with carbon nanotubes, a glassy carbon electrode modified with a metal nanoparticle, a glass strip modified with carbon nanotubes, a glass strip modified with a metal nanoparticle, a gold electrode modified with carbon nanotubes, or a gold electrode modified with a metal nanoparticle; and
b) a plurality of single-stranded DNA oligonucleotides adhered to the carbon SPE surface via Van der Waals forces, wherein at least a portion of the single-stranded DNA oligonucleotides are labeled with an electrochemical label.

11. The system of claim 10, wherein the single-stranded DNA oligonucleotides are single-stranded primers, or are single-stranded DNA amplicons comprising single-stranded primers.

12. The system of claim 11, wherein the single-stranded primers are from 10-50 nucleotides in length.

13. The system of claim 11, wherein the amplicons are from 80 to 120 nucleotides in length.

14. The system of claim 10, wherein the metal nanoparticle comprises gold, silver, iron, cobalt, platinum, carbon, or copper.

15. The system of claim 10, wherein the electrochemical label is selected from methylene blue, methylene violet, ruthenium hexamine, ferrocene, anthraquinone, an anthraquinone derivative, a metal nanoparticle, or a combination thereof.

16. A system comprising:
a) a carbon screen printed electrode (SPE); and
b) a plurality of single-stranded DNA oligonucleotides adhered to the carbon SPE surface via Van der Waals forces, wherein at least a portion of the single-stranded DNA oligonucleotides are labeled with an electrochemical label.

17. The system of claim 16, wherein the single-stranded DNA oligonucleotides are single-stranded primers or are single-stranded DNA amplicons comprising single-stranded primers.

18. The system of claim 17, wherein the single-stranded primers are from 10-50 nucleotides in length.

19. The system of claim 17, wherein the amplicons are from 80 to 120 nucleotides in length.

20. The system of claim 16, wherein the electrochemical label is selected from methylene blue, methylene violet, ruthenium hexamine, ferrocene, anthraquinone, an anthraquinone derivative, a metal nanoparticle, or a combination thereof.
